(19) United States
(12) Patent Application Publication
(10) Pub. No.: US 2018/0310876 A1
(43) Pub. Date: Nov. 1, 2018

(54) NONCONTACT DETECTION METHOD OF SLEEP STAGES AND SLEEP-DISORDERED BREATHING

(71) Applicant: SHANGHAI MEGAHEALTH TECHNOLOGIES CO., LTD, Shanghai (CN)

(72) Inventor: Ping LI, Shanghai (CN)

(21) Appl. No.: 15/770,326

(22) PCT Filed: Nov. 20, 2015
(86) PCT No.: PCT/CN2015/095213

§ 371 Date: Apr. 23, 2018

(30) Foreign Application Priority Data
Sep. 22, 2015 (CN) 201510605085.0

(51) Int. Cl. A61B 5/00; A61B 5/0205; A61B 5/05

(52) U.S. Cl. CPC A61B 5/4818; A61B 5/02405; A61B 5/0507; A61B 5/0205

(57) ABSTRACT

A noncontact method for detecting and monitoring sleep-disordered breathing includes the following steps: (1) during a test, a tested person lies in a bed, and the antennas of a wireless transceiver are aligned to the human body; (2) the wireless transceiver has the received signal to be subjected to digital signal processing, and mode recognition, and records and reports disordered events. Such non-contact measurement can simplify operation and facilitate long-term dynamic monitoring.

### TECHNICAL FIELD

The present invention is related to a noncontact detection method of sleep stages and sleep-disordered breathing, specifically related to a noncontact detection method of sleep stages and sleep-disordered breathing based on respiratory rate, breathing extend and heart rate variability monitoring.

### BACKGROUND

It has long been believed that sleep is a complete rest process necessary for a person to eliminate fatigue. However, tests on brain electrical activity of people and animals find that brain activity in a sleep stage is not at a stationary state, but shows a series of actively adjusted periodic changes, and at this time, a variety of physiological functions of the body, such as a sensory function, a motor function and an autonomic nerve function perform regular activities on different degrees with the change of the sleep depth. A universal international method is to divide the sleep into two different time phases according to brain electrical performance, eye movement situations and muscle tension changes in a sleep process, namely, non-rapid eye movement sleep (NREM) and rapid eye movement sleep (REM).

The non-rapid eye movement sleep (NREM) is characterized by progressing with the deepening of sleep beginning from the sleep at night. In this stage, human breath becomes shallow, slow and uniform, the heart rate slows down, the blood pressure drops, the muscles of the whole body relax (still can maintain a certain posture), and no obvious eye movement is made. This stage can be further divided into 4 periods, the first period is a hypnagogic period, the second period is a shallow sleep period, the third period is a moderate sleep period, and the fourth period is a deep sleep period.

The rapid eye movement sleep (REM): after sleeping for about 90 minutes, the human body enters rapid eye movement, and the rapid eye movement sleep is characterized by rapid eye rotation. In this stage, the sensory function of the human body is further reduced, the muscles are more relaxed, and tendon reflex disappears. At this time, the blood pressure is elevated compared with that in the non-rapid eye movement sleep, the breath is slightly faster and irregular, and the body temperature and the heart rate are also raised. In this stage, a variety of metabolic functions in vivo are significantly increased, so as to ensure the synthesis of brain tissue protein and supplement of consumed substances, promote normal development of the nervous system and accumulate energy for activities on the second day. Studies suggest that the NREM sleep is primarily a rest of the cerebral cortex, while the REM sleep is primarily a systemic rest.

Sleep apnea syndrome is a common disease which seriously harms physical health. The main incidence population is middle-aged men over 40 years old and the elderly over 60 years old. The symptoms of the sleep apnea syndrome is that an apnea event or a hypopnea event occurs for more than 30 times within a sleep cycle of 7 hours each day, or the two respiratory disorder events occur for more than 5 times within each hour. The apnea event refers to that no airflow passes through the respiratory tract within continuous 10 seconds in a sleep state. The hypopnea event refers to that the flow of respiratory airflow or the amplitude of the respiratory movement of the thorax and abdomen is smaller than 50% of a normal value in the sleep state.

At present, a common medical method is to test the respiratory rate by using a test pectoral girdle, test inspiratory capacity by using a respiratory duct and achieve comprehensive diagnosis in combination with electrocardiogram, electroencephalogram, myoelectricity and an oxyhemoglobin saturation measurement method. The traditional test and diagnostic methods need to use a variety of test instruments at the same time, on some occasions, the use is very inconvenient, initially tested patients are unaccustomed, thus the test results are influenced, and moreover, this method is not convenient for long-time monitoring for multiple times in multiple days.

### SUMMARY

Aiming to the defect in the existing art, the present invention proposes a noncontact system method for detecting and monitoring sleep-disordered breathing. The power of a wireless signal transmitted by this system method is very low and is within 20 mw, and thus being harmless to human body. Due to such noncontact measurement, the use is very convenient, and long-term dynamic monitoring and test are facilitated.

To achieve the above object, the present invention is realized by a noncontact method for detecting and monitoring sleep-disordered breathing, including the following steps: 1. during a test, the tested person lies in bed, and the antennas of the wireless transceiver are arranged within a certain range from the tested person. The receiving antenna and the sending antenna are aligned to the human body; 2. a wireless transmitter have the received signal be subjected to digital signal processing, and mode recognition, and then record and report the disordered events.

The sending antenna of the wireless transceiver is a customized ultra-wideband antenna, the sent signal is a narrow pulse with a width smaller than 15 ns, the smaller the pulse width is, the wider the frequency spectrum of the signal is, when this radio wave containing ultra-wideband pulses is directly irradiated to the human chest, the human thorax and abdomen will reflect the radio wave, and the reflected echo will change with time and carries periodic mechanical wave information caused by the respiratory movement of the thorax and abdomen and heartbeat. The receiving antenna is also an ultra-wideband antenna. The receiving antenna will input the received echo to the digital signal processing module after processing the same. The digital signal processing module is mainly used for recovering the respiratory wave signal, the body movement signal and the cardiac impulse signal through the digital signal processing method of weak signals. Then, these signals are input to the mode identification module, the mode identification module extracts signs at first and then performs mode identification with a pre-trained template, so as to dynamically detect the sleep stages and the start time, the end time and the duration of the sleep-disordered breathing event. The mode identification result is reported to superior equipment by the result recording and reporting software.

When a wireless transmitter directly irradiates ultra-wideband electromagnetic waves with central frequency ranging from 4 G to 10.5 G onto the thorax and abdomen of the human body, the skin, the endoskeletons and the fat of the viscera of the human body will reflect the electromagnetic waves to a certain extent according to the dielectric properties per se. Reflected electromagnetic waves received by a wireless receiver within a short distance are called echo. When the time width of the short-time pulse is of a nanosecond level, the transmitting waves and the echo are a frequency domain signal with a large bandwidth and have good temporal resolution.

When a wireless transmitter directly irradiates ultra-wideband electromagnetic waves with central frequency ranging from 1 G to 10.5 G onto the thorax and abdomen of the human body, the skin, the endoskeletons and the fat of the viscera of the human body will reflect the electromagnetic waves to a certain extent according to the dielectric properties per se. Reflected electromagnetic waves received by a wireless receiver within a short distance are called echo. When the time width of the short-time pulse is of a nanosecond level, the transmitting waves and the echo are a frequency domain signal with a large bandwidth and have good spatial resolution.

The effective detection distance of the system is 0.5 m to 10 m, the central frequency of the wireless signal is 1 G to 10 G, and the width of the narrow pulse is smaller than 15 ns.

In the present invention, a special customized transmitting antenna is used for transmitting an ultra-wideband wireless pulse signal with an absolute bandwidth larger than 1.5 GHz at 20 dB or a bandwidth larger than 25% of central frequency to directly irradiate the thorax and abdomen breathing positions of a tested person, periodic mechanical movements of human respiration and heartbeat reflect this wave to form an echo signal, a special customized wideband receiving antenna is used for receiving this echo signal. For the echo signal, a digital signal processing extraction method of weak signals is used for extracting a respiratory wave signal, a body movement signal and a cardiac impulse signal from the echo signal, then features are extracted, a model identification method containing a training information data template is used for dynamically detecting sleep stages and two sleep-disordered breathing events, namely, sleep apnea and sleep hypopnea, and the start time, the end time and the duration are reported.

The beneficial effect of the present invention: the power of a wireless signal transmitted by this system method is very low and is within 20 mw, and thus being harmless to human body. Due to such noncontact measurement, the use is very convenient, and long-term dynamic monitoring and test are facilitated.

### DESCRIPTION FOR THE DRAWINGS

The present invention is explained accompany with the drawing and detailed embodiments below.

FIG. 1 is the system block diagram of the present invention.

FIG. 2 is the mode recognition block diagram of the present invention.

### DETAILED EMBODIMENTS

To make the technical means, inventive feature, and the obtained object and effect, realized by the present invention be easily understand, the present invention is further illustrated combining with the detailed embodiments.

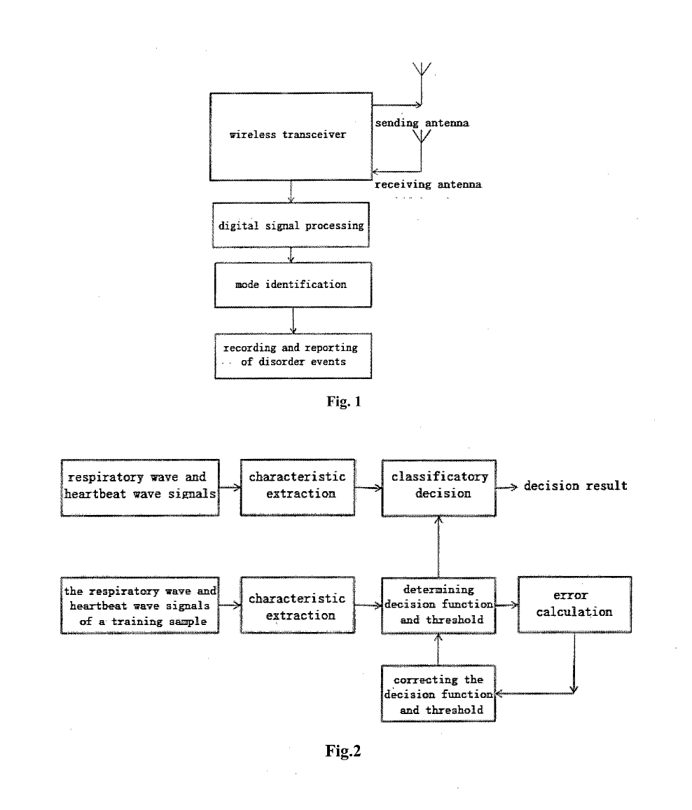

Refer to FIG. 1 to FIG. 2, the following technical solution is adopted in the present invention. A noncontact system method for detecting and monitoring sleep-disordered breathing includes a wireless transmitter, digital signal processing, mode recognition, and recording and reporting of the disordered event. A transmitting antenna and receiving antenna are provided on the wireless transmitter, and the wireless transmitter have the received signal be subjected to digital signal processing and mode recognition, and then record and report the disordered events at last.

In the detailed embodiment, at the transmitter, the collected echo intensity is subjected to an analog/digital conversion and a two-dimensional sampling sequence including slow time and fast time is obtained, and then the sampling sequence is input into a digital signal processing module. The effective detecting distance of the system is from 0.5 m to 3 m, and the mid-frequency of the wireless signal is from 4.2 G to 10 G, the narrow pulse width is from 1.5 ns to 5 ns. And then, by way of the the digital signal process, the respiratory wave and the heart beating wave is recovered. Since the power of the wireless signal employed is very low, the echo signal will be interfered by other RF signal and RF noise. So, decreasing the noise and improving shall be applied to the echo signal during the wireless transmission firstly. In the present system, the method of slow time and fast time average filtering is employed to remove the interference of the noise.

The respiratory frequency, the breathing extent, the body movement and the cardiac impulse signal of the human body correspondingly change in different sleep stages, and then the sleep stages can be determined according to these changes.

The sleep-disordered breathing events mainly include a sleep apnea event and a sleep hypopnea event. The former occurs in the sleep process, and the apnea event means that no airflow passes through the respiratory tract within continuous 10 seconds in a sleep state. The hypopnea event means that the flow of respiratory airflow or the amplitude of the respiratory movement of the thorax and abdomen is smaller than 50% of a normal value in the sleep state.

From the beginning to the end of the two events, the respiratory rate, the breathing extent, the heart rate and the heart rate variability change to a certain extent, and the statistical properties of these several life sign parameters will also change relevantly.

The sleep stages and the sleep-disordered breathing events are detected by a method of contrasting mode identification with a medical gold standard in the system. The block diagram of specific mode identification is as shown in FIG. 2.

Data of the training sample is composed of the respiratory wave signal using the medical gold standard, the cardiac impulse wave signal and the data of the actually measured sleep-disordered breathing events.

A decision function and a decision threshold obtained by training a large number of training samples are used for determining the dynamically input respiratory wave signal and the cardiac impulse wave signal and determining whether the sleep apnea event and the sleep hypopnea event occur within a certain time window. If detecting the events, then the start time, the end time and the duration are recorded.

A result recording and reporting module is a software module and includes a timer used for calculating an apnea-hypopnea index (Apnea-hypopnea Index, AHI), namely, the times of the apnea event and the hypopnea event within an hour on average according to the detected sleep stages and the sleep-disordered breathing events, for example, the occurrence time, frequency and the like.

Related results are reported to the superior equipment.

The superior equipment is responsible for recording, counting all the data and providing diagnosis and treatment services.

The above illustration and description describe the basic theory and main features of the present invention. The person in the art shall know that the present invention is not limited by the above embodiment. The content described in the above embodiment and specification is only for explaining the theory of the present invention. There are various of changes and improvements without deviating the spirit and range of the present invention. Such changes and improvements fall into the range of the present invention as claimed. The protection range as claimed by the present invention is defined by the attached claims and its equivalent.

1. A noncontact method for detecting and monitoring sleep-disordered breathing, including the following steps: (1). during a test, the tested person lies in bed, and the antennas of the wireless transceiver are arranged within a certain range from the tested person, the receiving antenna and the sending antenna are aligned to the human body; (2). a wireless transmitter have the received signal be subjected to digital signal processing, and mode recognition, and then record and report the disordered events.

2. A noncontact method for detecting and monitoring sleep-disordered breathing as described by claim 1, characterized in that, the sending antenna of the wireless transceiver is a customized ultra-wideband antenna; the sent signal is a narrow pulse with a width smaller than 15ns, the smaller the pulse width is, the wider the frequency spectrum of the signal is; when this radio wave containing ultra-wideband pulses is directly irradiated to the human chest, the human thorax and abdomen will reflect the radio wave, and the reflected echo will change with time and carries periodic mechanical wave information caused by the respiratory movement of the thorax and abdomen and heartbeat; the receiving antenna is also an ultra-wideband antenna; the receiving antenna will input the received echo to the digital signal processing module after processing the same; the digital signal processing module is mainly used for recovering the respiratory wave signal, the body movement signal and the cardiac impulse signal through the digital signal processing method of weak signals; then, these signals are input to the mode identification module, the mode identification module extracts signs at first and then performs mode identification with a pre-trained template, so as to dynamically detect the sleep stages and the start time, the end time and the duration of the sleep-disordered breathing event; the mode identification result is reported to superior equipment by the result recording and reporting software.

2. A noncontact method for detecting and monitoring sleep-disordered breathing as described by claim 1, characterized in that, when a wireless transmitter directly irradiates ultra-wideband electromagnetic waves with central frequency ranging from 1 G to 10.5 G onto the thorax and abdomen of the human body, the skin, the endoskeletons and the fat of the viscera of the human body will reflect the electromagnetic waves to a certain extent according to the dielectric properties per se; reflected electromagnetic waves received by a wireless receiver within a short distance are called echo; when the time width of the short-time pulse is of a nanosecond level, the transmitting waves and the echo are a frequency domain signal with a large bandwidth and have good spatial resolution.

3. A noncontact method for detecting and monitoring sleep-disordered breathing as described by claim 1, characterized in that, when a wireless transmitter directly irradiates ultra-wideband electromagnetic waves with central frequency ranging from 1 G to 10.5 G onto the thorax and abdomen of the human body, the skin, the endoskeletons and the fat of the viscera of the human body will reflect the electromagnetic waves to a certain extent according to the dielectric properties per se; reflected electromagnetic waves received by a wireless receiver within a short distance are called echo; when the time width of the short-time pulse is of a nanosecond level, the transmitting waves and the echo are a frequency domain signal with a large bandwidth and have good spatial resolution; the effective detection distance of the system is 0.5 m to 10 m, the central frequency of the wireless signal is 1 G to 10 G, and the width of the narrow pulse is smaller than 15ns.

4. A noncontact method for detecting and monitoring sleep-disordered breathing as described by claim 1, characterized in that, the result recording and reporting module is a software module and includes a timer used for calculating an apnea-hypopnea index according to the detected sleep stages and the sleep-disordered breathing events.
